(19) United States
(12) Patent Application Publication
(10) Pub. No.: US 2026/0198955 A1
(43) Pub. Date: Jul. 16, 2026

(54) TENSIONING ENDOSCOPIC DEVICES AND METHODS OF USE THEREOF

(71) Applicant: BOSTON SCIENTIFIC MEDICAL DEVICE LIMITED, Galway (IE)

(72) Inventors: Gagan Jain, Titilagarh (IN); Rahul Mathur, Gurgaon (IN); Jagadeeswar Reddy Uchala, Gurgaon (IN)

(73) Assignee: BOSTON SCIENTIFIC MEDICAL DEVICE LIMITED, Galway (IE)

(21) Appl. No.: 19/446,441

(22) Filed: Jan. 12, 2026

Related U.S. Application Data
(60) Provisional application No. 63/744,584, filed on Jan. 13, 2025.

(51) Int. Cl. A61B 17/32; A61B 1/00; A61B 1/018; A61B 17/00

(52) U.S. Cl. CPC A61B 17/320016; A61B 1/00119; A61B 1/00137; A61B 1/018; A61B 2017/00367; A61B 2017/00477

(57) ABSTRACT

The present disclosure relates generally to the field of medical devices. In particular, the present disclosure relates to endoscopes, and methods of use thereof, for endoscopic procedures such as tissue dissection. For example, an endoscope for tissue dissection can comprise a cap attachable to a distal end of an endoscope having a working channel extending therethrough, the cap including lumens that are coextensive with the working channel; a tensioning member disposable within the working channel and a first lumen of the lumens, wherein the tensioning member includes: a distal tip; and an actuation mechanism that is coupled to the distal tip and is configured to translate the distal tip longitudinally relative to at least the cap; and an endoscopic instrument disposable within the working channel and a second lumen of the lumens.

### CROSS-REFERENCE TO RELATED APPLICATIONS

The application claims the benefit of U.S. Provisional Patent Application Serial No. 63/744,584, filed on January 13, 2025, the disclosure of which is incorporated herein by reference.

### FIELD

The present disclosure relates generally to the field of medical devices. In particular, the present disclosure relates to tensioning endoscopic devices and methods of use thereof, for endoscopic procedures such as tissue dissection.

### BACKGROUND

Various endoscopic surgical procedures require maneuvering about various anatomical structures. Some procedures, such as endoscopic mucosal resection (EMR), Endoscopic Submucosal Dissection (ESD), Pre-Oral Endoscopic Myotomy (POEM), etc., allow for minimally invasive endoscopic removal of benign and early malignant lesions, such as in the gastrointestinal (GI) tract. Minimally-invasive surgical techniques like these typically allow for faster recovery than with open or laparascopic surgical procedures. However, because such procedures are minimally invasive, there is limited space to maneuver within the body, and such procedures typically require a high degree of expertise.

For example, ESD is a minimally invasive procedure that enables en bloc endoscopic resection of superficial tumors in the gastrointestinal tract. During ESD procedures, medical professionals utilize an endoscope to access and visualize target tissue sites while deploying various medical devices through the endoscope's working channel to perform the dissection. The procedure typically involves using an endoscope equipped with visualization capabilities, such as a light and/or camera, to illuminate and view the target tissue area. Through the working channel of the endoscope, physicians can deploy different medical instruments including cutting devices, grasping tools, and tissue traction devices to assist with the procedure.

Tissue traction may be an important aspect of ESD procedures, as it helps provide proper visualization of the submucosal layer during dissection. For instance, performing an endoscopic tissue resection/dissection procedure may include maintaining traction as the boundaries of the target tissue are dissected for accuracy and efficiency of the procedure. Various traction methods and devices have been developed to help maintain appropriate tension on the target tissue during these procedures.

Physicians may use a tethered system in which an endoscopic clip is attached to a length of filament extending external to the patient to retract/immobilize a target tissue for dissection along the tissue margins and/or to retrieve the dissected target tissue for biopsy. Such tethered systems may be unable to maintain or adjust tension applied to the target tissue, possibly obstructing a medical professional’s view of the target tissue and/or interfering with accessory tools. These complications may directly contribute to increased procedures time, complexity, and risk of perforation or bleeding.

It is with the above considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

Some current ESD procedures typically use Multi-loop Traction Devices (MLTD). The MLTD can be comprised of three connected rings e.g., which are each made of a linear biocompatible low-density polyethylene plastic. The MLTD can be delivered to the lesion site from the forceps channel of an endoscope. The MLTD is intended to be cut and removed by grasping one of the rings using biopsy forceps and pulling away. However, use of MLTD presents several challenges or drawbacks. For instance, the MLTD loop can be prone to breaking (e.g., in vivo) when it gets caught in the proximal end of the jaw during opening and closing actions. The MLTD can be prone to falling off or otherwise becoming inadvertently dislodged off the distal end of an endoscopic device (e.g., the jaw) in vivo. The breakage and/or inadvertent dislodging of the MLTD can result in added time or difficulty such as requiring introduction of a new MLTD device and prolonging the procedure. Moreover, even when the MLTD is successfully deployed in vivo, clips used to attach the MLTD e.g., to healthy tissue must be left behind in the patient's body as they cannot be safely removed. Further, use of the MLTD can inherently result in poor visualization and/or inadequate tissue manipulation (e.g., as tensioning and/or location of the MLTD cannot be adjusted once the MLTD is attached via clips or other attachment mechanism to tissue.

Accordingly, the present disclosure provides a novel tensioning member device for ESD procedures. The tension endoscopic devices herein can include a distal cap. The distal cap can include openings or orifices configured to permit a tensioning member and a cutting knife to extend therethrough. For instance, the tensioning member that can be inserted through a working channel (e.g., a 4.2 millimeter working channel) alongside a cutting knife. The tensioning member can include a distal tip that can be coupled (e.g., interference fit or pressed against) to target tissue (e.g., lesion). For instance, the tensioning member can be extended distal to distal cap of the endoscopic device and can be coupled to a screw mechanism. Actuation of the screw mechanism or similar mechanism can cause the tensioning member to longitudinally translate (e.g., move distally) relative at least to a distal cap thereby imparting a tensioning force on target tissue. The amount of force imparted by the tensioning member is adjustable. For instance, a proximal knob can be rotated clockwise or counterclockwise to control forward/backward movement of the tensioning member.

For example, in some embodiments, the endoscope or duodenoscope is inserted to access the lesion site, the tensioning member and ESD knife are inserted through the working channel, where the tensioning member is insertable through the distal cap. In such instances, the distal tip can contact the lesion e.g., via rotation of the screw mechanism such that the distal tip translates distally until the distal tip is in contact with the lesion. Additionally, the proximal knob is rotated to adjust tension as needed during dissection. For example, the proximal knob can be rotated to maintain the distal tip (e.g., move the distal tip from a first (initial) position in contact with the lesion to a second position that is more distal relative to the first position to maintain contact with the lesion as portions (e.g., proximal regions) of the lesion are removed.

Maintaining contact of the distal tip with the lesion (e.g., a proximal surface of the lesion) during the dissection procedure (e.g., when dissecting tissue) can reduce complications, reduce an amount of time of the procedure, and/or improve visibility throughout the procedure as compared to other approaches such as those that employ a MLTD. For instance, the devices and methods herein can permit simultaneous cutting and precise tension control of the distal tip of the tensioning member. Moreover, unlike other approaches such as those that employ a MLTD, the devices and methods herein can be employed with an absence of clips and thus avoid issues (e.g., leaving foreign matter such as the clips behind in vivo) and/or complexities associated with employing multiple device exchanges. Thus, the devices and methods herein can yield faster and safer procedures (e.g., ESD procedures), while also addressing the limitations of current approaches such as those that employ MLTD.

In an aspect, an endoscope for tissue dissection is provided. The endoscope comprising: a cap attachable to a distal end of an endoscope having a working channel extending therethrough, the cap including lumens coextensive with the working channel; a tensioning member disposable within the working channel and a first lumen of the lumens, wherein the tensioning member includes: a distal tip; and an actuation mechanism that is coupled to the distal tip and is configured to translate the distal tip longitudinally relative to at least the cap; and an endoscopic instrument disposable within the working channel and a second lumen of the lumens.

In some aspects, which may be used in conjunction with the other aspects herein, wherein a distal end of the distal tip is configured at an angle to a body portion of the distal tip.

In some aspects, which may be used in conjunction with the other aspects herein, wherein the distal end of the distal tip is a pointed terminus.

In some aspects, which may be used in conjunction with the other aspects herein, wherein the actuation mechanism is a screw mechanism, and wherein the endoscope further includes a knob coupled to a proximal end of the screw mechanism that is configured to rotate the screw mechanism.

In some aspects, which may be used in conjunction with the other aspects herein, wherein the distal cap has a substantially planar distal end, and wherein the lumens extend through the substantially planar distal end of the distal cap.

In some aspects, which may be used in conjunction with the other aspects herein, wherein the lumens extend in a substantially longitudinal direction entirely through the distal cap and are in fluid communication with the working channel.

In some aspects, which may be used in conjunction with the other aspects herein, wherein the lumens each have a respective diameter that is less than a diameter of the working channel.

In some aspects, which may be used in conjunction with the other aspects herein, wherein the lumens are uninterrupted circular lumens.

In some aspects, which may be used in conjunction with the other aspects herein, wherein the distal tip is non-movably coupled to the actuation mechanism.

In some aspects, which may be used in conjunction with the other aspects herein, the distal tip is configured to translate longitudinally a first distance relative to a distal end of the distal cap, wherein the first distance is in a range from about 5 millimeters to about 15 millimeters; and a cutting tip of the endoscopic instrument is configured to translate longitudinally a second distance from the distal end of the distal cap, wherein the second distance is in a range from about 5 millimeters to about 15 millimeters.

In some aspects, which may be used in conjunction with the other aspects herein, wherein a diameter of the first lumen is substantially equal to a diameter of a proximal end the distal tip, a diameter of the actuation mechanism, or both.

In some aspects, which may be used in conjunction with the other aspects herein, wherein a diameter of the second lumen is substantially equal to a diameter of a body portion of the endoscopic instrument.

In some aspects, which may be used in conjunction with the other aspects herein, wherein the first lumen is spaced a distance from the second lumen.

In some aspects, which may be used in conjunction with the other aspects herein, wherein the distance is in a range from about 0.1 millimeters to about 1.0 millimeters.

In some aspects, which may be used in conjunction with the other aspects herein, wherein the working channel has an internal diameter of about 4.2 millimeters and wherein the distance is in a range from about 0.1 millimeters to about 0.5 millimeters.

In another aspect, an endoscope for tissue dissection is provided. The endoscope comprising: a distal cap attachable to a distal end of an endoscope having a working channel extending therethrough, the cap including lumens coextensive with the working channel, wherein the lumens include a first lumen and a second lumen that is spaced apart radially from the first lumen; a tensioning member disposable within the working channel and a first lumen of the lumens, and wherein the tensioning member includes: a distal tip including a distal end; and a rotatable actuation mechanism that is non-movably coupled to a proximal end of the distal tip and is configured to rotate relative to a longitudinal axis of the endoscope to cause the distal tip to translate longitudinally relative to at least the distal cap; and a cutting knife including a cutting tip, wherein the cutting knife is disposable within the working channel and a second lumen of the lumens.

In another aspect, a method of endoscopic submucosal dissection is provided. The method comprising: inserting an endoscope in vivo to access a target lesion in a patient, wherein the endoscope includes a working channel and a distal cap coupled to a distal end of the endoscope, and wherein the distal cap includes a plurality of a lumens that are coextensive with a working channel; distally advancing a tensioning member through the working channel and a first lumen of the

plurality of lumens of the distal cap to from a first position where a distal tip of the tensioning member is not in contact with the target lesion to a second position where the distal tip of the tensioning member is in contact with the target lesion; distally advancing a cutting knife through the working channel and a second lumen of the plurality of lumens to from a first position where a cutting tip of the cutting knife is not in contact with the target lesion to a second position wherein the cutting tip of the cutting knife is in contact with the target lesion; and actuating the cutting tip to dissect at least a portion of target lesion while the cutting tip is in the second position and the distal tip of the tensioning member is in the second position.

In some aspects, which may be used in conjunction with the other aspects herein, further comprising: rotating a proximal knob a first direction to distally advance the distal tip of the tensioning member to the first position; and subsequent to actuating the cutting tip to cut at least a portion of target lesion, rotating the proximal knob the first direction to distally advance the tensioning member to a third position that is distal to the second position and is in contact with a remainder of the target lesion.

In some aspects, which may be used in conjunction with the other aspects herein, further comprising: actuating a handle to distally advance the cutting tip of the cutting knife to the second position; subsequent to advancing the tensioning member to the third position, actuating the handle to distally advance the cutting tip of the cutting knife from the second position to a third position that is distal to the second position; and actuating the cutting tip while the cutting tip is in the third position and the distal tip of the tensioning member is in the third position to cut at least a portion of the remainder of the target lesion.

In some aspects, which may be used in conjunction with the other aspects herein, further comprising retracting the endoscope to remove the distal cap, the tensioning member, and the cutting knife from the patient after completing dissection of the target lesion, wherein substantially no materials are left in the patient after retracting the endoscope from the patient.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1A provides a perspective view of an example endoscope in a first configuration disposed in vivo in a patient with a target lesion according to an embodiment of the present disclosure;

FIG. 1B provides a perspective view of the example endoscope of FIG. 1A in a second configuration;

FIG. 1C provides a perspective view of the example endoscope of FIG. 1A in a third configuration;

FIG. 1D provides a perspective view of the example endoscope of FIG. 1A in a fourth configuration;

FIG. 1E provides a perspective view of the example endoscope of FIG. 1A in a fifth configuration;

FIG. 1F provides a perspective view of an example endoscope of FIG. 1A in a sixth configuration;

FIG. 2 provides another view of the example endoscope of FIG. 1A; and

FIG. 3 provides an example of a method of endoscopic tissue dissection according to an embodiment of the disclosure.

### DETAILED DESCRIPTION

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to endoscopic systems and methods designed to provide traction within the gastrointestinal tract during a tissue dissection/resection procedure, it should be appreciated that such systems and methods may be used to manipulate a variety of tissues within a variety of different body lumens and/or body passages in conjunction with or independent of an endoscope.

As used herein, the singular forms “a,” “an,” and “the” are intended to include the plural forms as well, unless the context clearly indicates otherwise. It will be further understood that the terms “comprise”, “comprises”, and/or “comprising,” or “includes”, and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

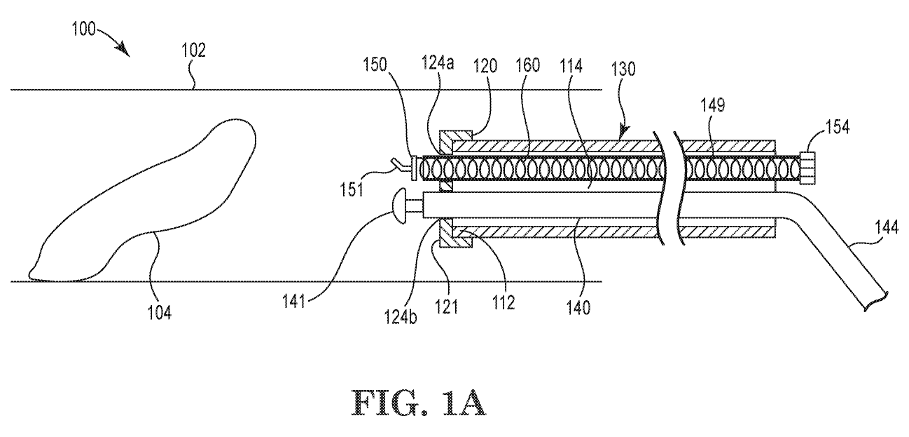

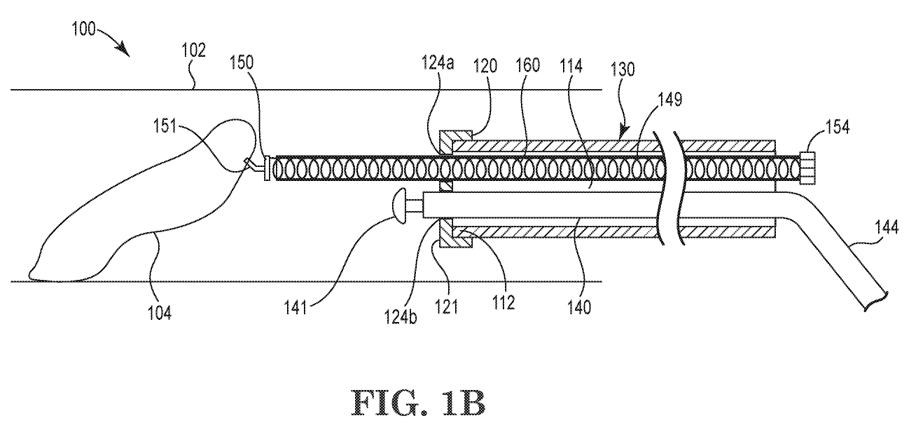

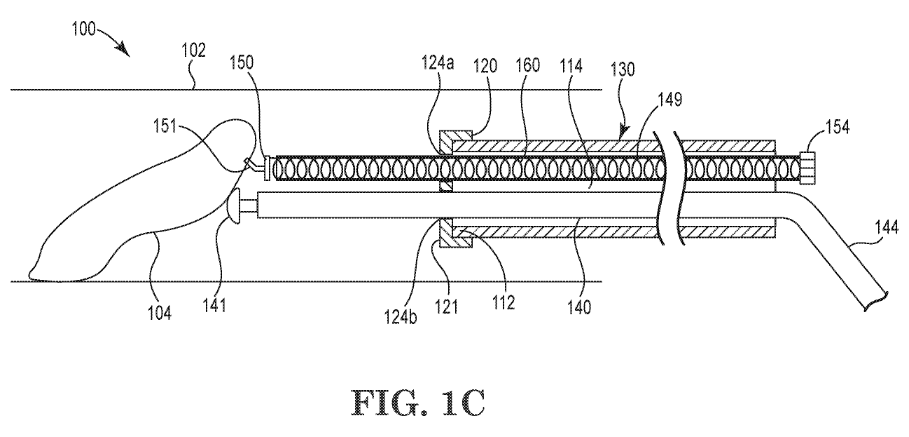

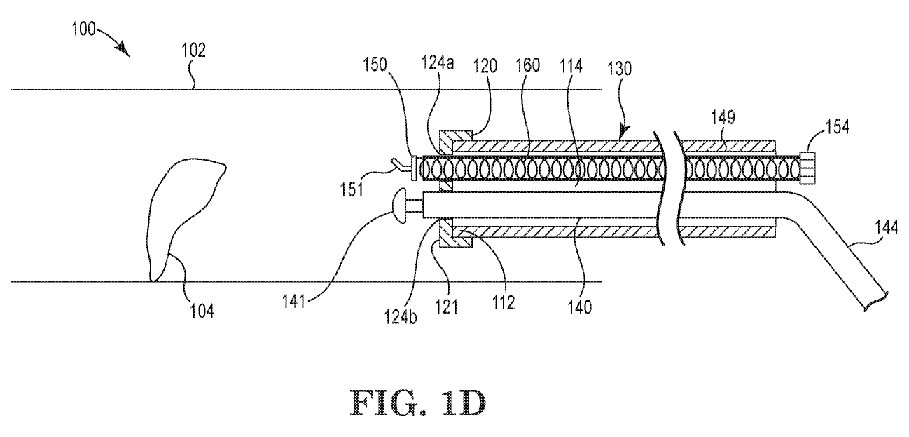

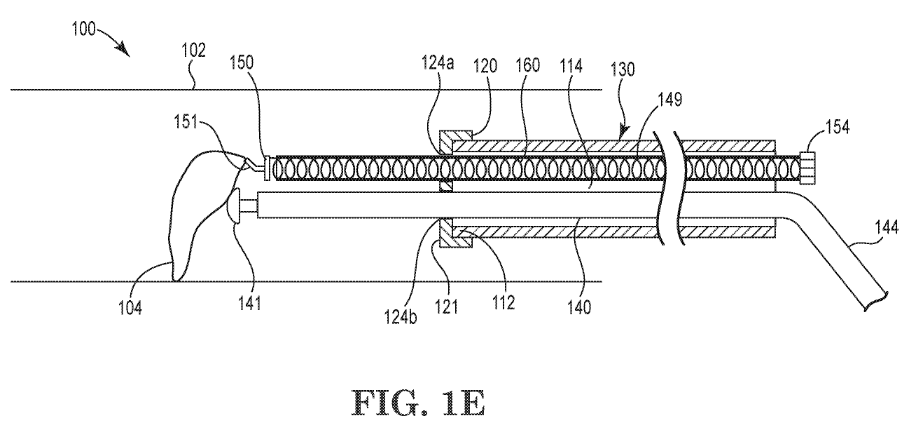

Referring to FIGS. 1A-1F, in one embodiment, an example of a system 100 includes an endoscope 130 disposed in vivo. That is, the endoscope 130 can be positioned within the vasculature or a body cavity of patient to facilitate various medical procedures such as those described herein. For instance, the endoscope 130 can be a duodenoscope that is configured for endoscopic submucosal dissection (ESD). For example, the endoscope 130 can be navigated within an esophagus 102 of a patient to permit removal of target tissue 104 (e.g., a lesion) located in the esophagus 102. In some instances, the target tissue 104 can be a target lesion that is disposed in vivo.

As illustrated in FIGS. 1A-1F, the endoscope 130 may include a cap 120 (e.g., endoscopic cap, etc.) attachable to (e.g., by a friction or interference fit, a threaded fit, a snap-lock fit, etc.) a distal end 112 of a flexible elongate tubular member (e.g., endoscope 130, etc.). The flexible elongate tubular member (e.g., endoscope 130) can have a proximal end (opposite the distal end 112), the distal end 112, and a working channel 114 extending between the proximal end and the distal end 112. The cap 120 can be removably or non-removably coupled to the distal end 112 of the endoscope 130. As illustrated in FIGS. 1A-1F, the distal cap 120 can overlay or entirely cover the distal end 112 of the endoscope 130. The working channel 114 represents one or more substantially longitudinally extending channels. For instance, the working channel 114 can have a substantially uniform diameter (e.g., 4.2 millimeters) taken along an entire length thereof.

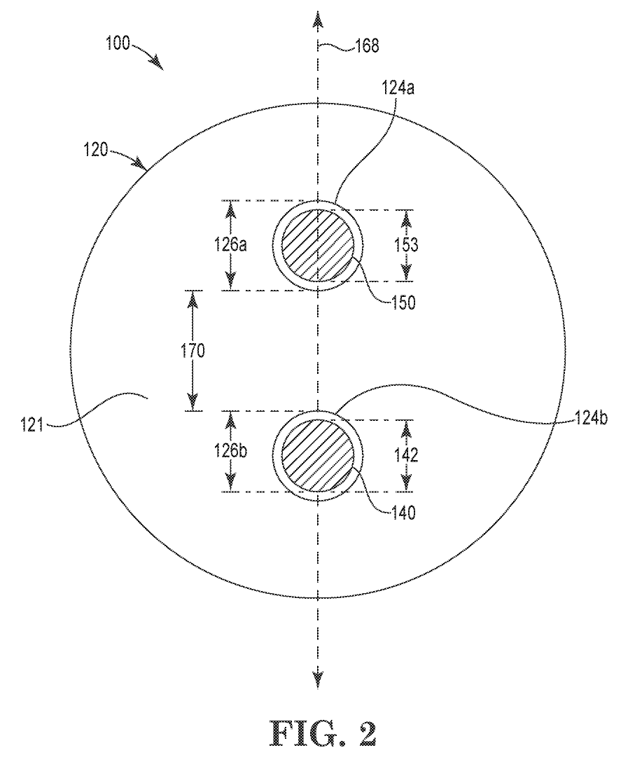

The distal cap 120 can include lumens such as a first lumen 124a and a second lumen 124b (collectively referred to herein as lumens 124). While illustrated as including two lumens 124, the distal cap 120 can include a different total quantity of lumens (e.g., three lumens, four lumens, etc.). The lumens 124 can extend in a substantially longitudinal manner through the distal cap 120. For instance, the lumens can extend entirely through the distal cap 120 (e.g., between a proximal end and the distal end 121 of the distal cap 120). In some embodiments, the distal cap 120 can be manifested as a distal cap having a substantially planar distal end 121, and the lumens 124 can extend through the substantially planar distal end 121 of the distal cap 120. As detailed herein (e.g., with respect to FIG. 2) the lumens can be spaced apart (e.g., radially spaced apart) a given distance. Having the lumens 124 be spaced a distance apart can promote aspects herein such as permitting a tensioning member to translate independent of an endoscopic instrument 140 (e.g., a cutting member) and/or can ensure that the tensioning member does not become entangled with other otherwise contact the endoscopic instrument.

The lumens 124 defined by the cap 120 may be coextensive with (e.g., aligned with) the working channel 114. Hence, the lumens 124 can be in fluid communication with the working channel 114. The lumens 124 may each have a respective diameter that is less than a diameter of the working channel 114. The lumens 124 may be uninterrupted lumens (e.g., that do not include any elements therein e.g., prior to the endoscopic instrument 140 and/or the tensioning member 149 being disposed in the lumens 124, as detailed herein.

In some embodiments, the lumens 124 can be configured with a shape and a size that is substantially equal to a shape and size (e.g., an outer diameter) of a corresponding element that is configured to be disposed in the lumens 124. For instance, in some embodiments, a diameter of the first lumen 124a may be substantially equal to a diameter of a proximal end of the distal tip 150, a diameter of an actuation mechanism 160, or both. For example, the diameter (e.g., diameter 153 as illustrated in FIG. 2) of the first lumen 124a can be substantially equal to a diameter (e.g., 126a) that is a largest taken at any point longitudinal along the distal tip) of the distal tip 150, as illustrated in FIGS. 1A-1E.

Similarly, in some embodiments, a diameter (e.g., diameter 142 as illustrated in FIG. 2) of the second lumen 124b may be substantially equal to a diameter (e.g., 126b) of a body portion of the endoscopic instrument 140. Employing lumens 124 with a shape and size that is substantially equal to a shape and a size of the corresponding element that is configured to the disposed in the respective lumens 124 can promote aspects herein such as mitigating or negating any radial movement of the corresponding elements (e.g., the endoscopic instrument 140 and/or the tensioning member 149) within the endoscope 130. Mitigating or negating the radial movement can ensure that the endoscopic instrument 140 and the tensioning member 149 do not become entangled, particularly when the distal tip 150 of the tensioning member 149 and the cutting tip of the endoscopic instrument 140 are positioned a distance distal to the distal end 121 of the distal cap 120, among other benefits such as ensuring that the tensioning member 149 imparts consistent tension on a lesion, etc.

In some embodiments, the lumens 124 can be substantially circular lumens having a smooth and uniform diameter surface. Employing circular lumens such as those with a smooth and uniform diameter surface (e.g., taken at any point longitudinally along a surface of the lumens 124) can promote aspects herein such as permitting the endoscopic instrument 140 and/or the tensioning member 149 to readily translate in a longitudinal manner and/or permitting rotation of the tensioning member 149 and/or the endoscopic instrument 140. For instance, an endoscopic instrument 140 may be movably/slidably disposable within the working channel 114 and an individual lumen of the lumens 124.

For example, the endoscopic instrument 140 can be configured to translate in a substantially longitudinally manner responsive to actuation of a handle 144. That is, the endoscopic instrument 140 for use with an endoscope 130 of the present disclosure may include a handle 144 that is operatively attached to a proximal end of the endoscopic instrument. In some embodiments the endoscopic instrument 140 can be configured to rotate (e.g., about or relative to a longitudinal axis or the endoscope 130) responsive to actuation or movement of the handle 144. While a handle 144 is illustrated, employing additional or alternate types of actuation mechanisms such as knobs, levers, etc. is possible. For instance, endoscopic instrument 140 can be a cutting member with a cutting tip (distal tip) where the cutting member can be distally translated (e.g., relative to the distal cap 120) to contact the target tissue 104 located distal to the distal cap 120. In some embodiments, the cutting member can be moved (e.g., rotated, etc.) while the cutting tip 141 is in contact with the target tissue 104 to dissect the target tissue 104.

The endoscopic instrument 140 can be a cutting member, as detailed herein, that is movably/slidably disposed in the working channel 114 and the second lumen 124b. While FIGS. 1A-1F illustrate the endoscopic instrument 140 as a cutting member, the use of other types of endoscopic instruments (e.g., grasping members, etc.) is possible. A tensioning member 149 may be movably/slidably disposable within the working channel 114 and an individual lumen of the lumens 124. For instance, the tensioning member 149 may be movably/slidably disposed in the working channel 114 and the first lumen 124a.

As illustrated in various figures herein, the endoscopic instrument 140 and the tensioning member 149 may each be extendable distally beyond the distal end 121 of the cap 120, e.g., to engage a target tissue 104. For instance, the distal tip 150 of the tensioning member 149 can be configured to translate longitudinally a first distance relative to the distal end 121 of the distal cap 120. The first distance may be in a range from about 5 millimeters to about 15 millimeters or from about 10 millimeters to about 15 millimeters.

In some embodiments, the cutting tip 141 of the endoscopic instrument 140 may be configured to translate longitudinally a second distance from the distal end 121 of the distal cap 120. The second distance may be in a range from about 5 millimeters to about 15 millimeters or from about 10 millimeters to about 15 millimeters. In some embodiments, the first distance can be substantially equal to the second distance. Having the first distance be substantially equal to the second distance can promote aspects herein, such as permitting the distal tip 150 (e.g., a distal end thereof) of the tensioning member 149 to remain in contact with and apply a tensioning force (e.g., substantially longitudinally directed force) to the target tissue 104 while the cutting tip 141 of the endoscopic instrument 140 is in contact with and dissects or cuts at least a portion of the target tissue 104.

In some embodiments, a distal end 151 of the distal tip 150 can be configured at an angle (a non-zero angle) to a body portion 152 of the distal tip. The angle can be any angle in a range from about 5 degrees to about 130 degrees. All individual values and sub-ranges from about 5 degrees to about 130 degrees are included. For instance, the distal tip 150 can be a hooked or arced distal tip. For instance, as illustrated in FIG. 1A, the distal tip 150 can be a hooked distal tip with the distal end 151 of the distal tip 150 configured at about a 90-degree angle relative to the body portion 152 of the distal tip 150, among other possibilities. Employing a hooked distal tip can promote aspects herein such as promoting the distal tip 150 to pierce and/or remain in contact with the target tissue 104, for instance, while the cutting tip 141 of the cutting member 140 dissects at least a portion of the target tissue 104.

In some embodiments, the distal end 151 of the distal tip 150 can form a pointed (e.g., sharp) terminus. For instance, the distal end 151 of the distal tip 150 can be manifested as a beveled edge, as illustrated in FIG. 1A. However, the distal end 151 of the distal tip 150 can take the form of various other types of pointed terminus. Having the distal end of the distal end 151 of the distal tip 150 be manifested as a pointed terminus can promote aspects herein such as promoting the distal tip 150 to pierce and/or remain in contact with the target tissue 104, for instance, while the cutting tip 141 of the cutting member 140 dissects at least a portion of the target tissue 104. For instance, in some embodiments, the distal end 151 of the distal tip 150 can be a pointed terminus and the distal end 151 of the distal tip 150 can be a hooked distal tip, for instance, as illustrated in FIG. 1A.

As mentioned, the distal tip 150 can be coupled to an actuation mechanism 160. For instance, a proximal end of the distal tip 150 can be coupled to a distal end of the actuation mechanism 160. The actuation mechanism 160 can thus be configured to permit that distal tip 150 to move longitudinally (e.g., translate in a substantially longitudinal manner) and/or can be configured to permit the distal tip 150 to rotate (e.g., rotate about or relative to a longitudinal axis of the endoscope 130). For example, the distal tip 150 can be coupled to the actuation mechanism 160 in a non-movable manner (e.g., welded, adhesively bonded, friction fit, etc.) such that the distal tip 150 does not move relative to the actuation mechanism 160.

Thus, any movement of the actuation mechanism 160 can impart a corresponding movement (e.g., in terms of magnitude and direction) of the distal tip 150. For example, the actuation mechanism 160 can be a screw mechanism, as illustrated in FIG. 1A. Thus, actuation of a knob 154 that is coupled to a proximal end of the actuation mechanism 160 can impart or otherwise cause rotation and translation (e.g., distally or proximally depending on a direction of rotation of the knob 154) of the distal tip 150. However, in some embodiments, the distal tip 150 can be configured to move (e.g., with at least one degree of freedom) relative to the actuation mechanism 160. Alternatively, or in addition, in some embodiments the distal tip 150 can be integral with (e.g., formed of the same material) as the actuation mechanism 160. While the actuation mechanism 160 is illustrated as a screw mechanism, other types of actuation mechanisms such as ratcheting or sliding-based actuation mechanisms are possible.

As mentioned, FIG. 1A provides a perspective view of the example endoscope 130 in a first (e.g., initial) configuration disposed in vivo in a patient with a target lesion (e.g., target tissue 104. As illustrated in FIG. 1A, the endoscope 130 can be disposed in in vivo in a vasculature or body cavity (e.g., the esophagus 102) of the patient. When in the first configuration, the entire endoscope 130 can be located proximal to (e.g., no portion or component of the endoscope 130 is contact with) the target tissue 104. For instance, each of the tensioning member 149 and the endoscopic instrument 140 can be disposed in or distal to the endoscope 130 at respective positions that are proximal to the target tissue 104 when the endoscope is in the first configuration.

FIG. 1B provides a perspective view of the example endoscope 130 in a second configuration disposed in vivo in the patient with the target tissue 104. As mentioned, each of the tensioning member 149 and the endoscopic instrument 140 can be configured to move (e.g., longitudinally translate) along the working channel of the endoscope 130 and the lumens of the distal cap 120. For instance, the tensioning member 149 can be translated distally via actuation

(rotation) of the proximal knob 154. While a proximal knob 154 is illustrated, employing additional or alternate types of actuation mechanisms such as handles, levers, etc. is possible. Thus, the tensioning member 149 can be moved from the first position, as illustrated in FIG. 1A, to a second position that is distal to the first position, as illustrated in FIG. 1B. When in the second position, the distal tip 150 of the tensioning member 149 can contact the target tissue. For instance, a distal end 151 of the distal tip 150 can pierce otherwise be located within the target tissue 104, in some embodiments. Having the distal tip 150 contact (e.g., pierce) the target tissue 104 can impart a force (e.g., a substantially distally extending force) of the target tissue 104. Stated differently, the distal tip 150 can maintain contact with and hold at least a proximal portion of the target tissue 104, as illustrated in FIG. 1B.

FIG. 1C provides a perspective view of the example endoscope 130 in a third configuration disposed in vivo in a patient with the target tissue 104 according to an embodiment of the present disclosure. As mentioned, the endoscopic instrument 140 (e.g., a cutting member) can be translated distally via actuation of the proximal knob 154. Thus, the endoscopic instrument 140 can be moved from the first position (e.g., where the cutting tip of the endoscopic instrument is not in contact with and is proximal to a target lesion), as illustrated in FIG. 1A, to a second position that is distal to the first position (e.g., where the cutting tip of the endoscopic instrument is in contact with the target lesion), as illustrated in FIG. 1B. In such instances, the cutting tip can remove or dissect at least a portion of the target tissue. For instance, the cutting tip can remove of dissect at least a proximal portion of the target tissue while cutting tip 141 of the endoscopic instrument 140 is in the second position and the distal tip 150 of the tensioning member 149 is in the second position, as illustrated in FIG. 1C.

FIG. 1D provides a perspective view of the example endoscope in a fourth configuration disposed in vivo in the patient with a remainder of the target lesion according to an embodiment of the present disclosure. That is, FIG. 1D illustrates the target tissue 104 having a portion (e.g., proximal portion) removed. Hence, due to removal of at least a portion of the target tissue 104, the entire endoscope 130 can be located proximal to (e.g., is not in contact with) the target tissue 104. For instance, each of the tensioning member 149 and the endoscopic instrument 140 can be disposed in the endoscope 130 at respective positions (e.g., respective second positions) that are proximal to the target tissue 104, namely a remainder or portion of the target tissue 104 that remains after the initial dissection of a proximal portion of the target tissue. As mentioned, maintaining tension on the target tissue 104 while dissecting the target tissue 104 can be desirable.

As such, FIG. 1E provides a perspective view of the example endoscope 130 in a fifth configuration disposed in vivo in a patient with the remainder of the target tissue 104 according to an embodiment of the present disclosure. That is, the tensioning member 149 (e.g., the distal tip 150 of the tensioning member 149) can be translated distally via further via rotation of the proximal knob 154. For instance, subsequent to actuating the cutting tip 141 to cut at least a portion of target tissue 104, the proximal knob 154 can be rotated in the first direction to distally advance the tensioning member 149 to a third position that is distal to the second position and is in contact with a remainder of the target tissue 104, as illustrated in FIG. 1E.

FIG. 1F provides a perspective view of an example endoscope 130 in a sixth configuration disposed in vivo in the patient according to an embodiment of the present disclosure. As illustrated in FIG. 1F, the endoscopic instrument 140 can be moved from the second position (e.g., where the cutting tip of the endoscopic instrument is not in contact with and is proximal to a remainder of the target tissue 104), as illustrated in FIG. 1D, to a third position (e.g., where the cutting tip 141 is in contact with the remainder to the target tissue 104). In such instances, the cutting tip can remove or dissect at least a portion of the remainder of the target tissue 104 once positioned at the third position. For instance, the cutting tip can remove or dissect at least a proximal portion of the remainder of the target tissue 104 while cutting tip 141 of the endoscopic instrument 140 is in the third position and the distal tip 150 of the tensioning member 149 is in the third position, as illustrated in FIG. 1F.

While FIGS. 1A-1F illustrate the endoscopic instrument 140 and the tensioning member 149 as being moved independently, in some embodiments the endoscopic instrument 140 and the tensioning member 149 can be moved simultaneously (e.g., translated distally simultaneously). As detailed herein, the distal tip 150 and the cutting tip 141 can be rotated independently (e.g., via rotation of the proximal knob or via rotation or actuation of the handle). For instance, in some embodiments the cutting tip 141 can be rotated to dissect the target tissue 104, while the distal tip 150 can remain in the same position (e.g., is not rotated).

FIG. 2 provides another view of the example endoscope 130 according to an embodiment of the present disclosure. That is, FIG. 2 illustrates and end on view taken from the perspective of the distal end of the endoscope 130. As mentioned, the endoscope 130 can include the cap 120 having a planar distal surface 121 and with the lumens 124 such as the first lumen 124a and the second lumen 124b extending through the cap 120. The lumens 124 can have substantially the same shape. For instance, the lumens 124 can each be substantially circular elongate lumens, as illustrated in FIG. 2. The lumens 124 can be spaced a distance from a periphery or radial most surface of the cap 120.

The lumens 124 can be the same size or different sizes (e.g., having the same inner luminal diameter or different inner luminal diameters. For instance, in some embodiments the lumens 124 can have different diameters. For example, the first lumen 124a can have a first diameter 153 that is different than a second diameter 142 of the second lumen 124b. In such embodiments, the first diameter 153 can be less than the second diameter 142. The first diameter 153 can be in a range from about 0.4 millimeters to about 1.4 millimeters and the second diameter 142 (e.g., about 2.0 millimeters) can be in a range from about 1.5 millimeters to about 2.2 millimeters, among other possible values. In some embodiments, the first lumen 124a and the second lumen 124b can be aligned along a common axis 168 that extends at a normal angle relative to a longitudinal axis of the endoscope 130, among other possible configurations.

As illustrated in FIG. 2, the lumens 124 can be spaced apart relative to each other. For instance, the first lumen 124a can be spaced a distance 170 apart from the second lumen 124b, as illustrated in FIG. 2. The distance 170 can be in a range from about 0.1 millimeters to about 1.0 millimeters. All individual values and sub-ranges from about 0.1 millimeters to about 1.0 millimeters are included. For instance, the distance 170 can be about 0.1 millimeters, about 0.2 millimeters, about 0.3 millimeters, about 0.4 millimeters or about 0.5 millimeters. Having the lumens 124 be spaced the distance 170 apart can promote aspects herein such as permitting the tensioning member 149 and/or the actuation mechanism 160 to readily move (e.g., rotate and/or translate) within the lumens 124 and/or mitigating any contact between the tensioning member 149 and/or the endoscopic instrument 140. For instance, having the lumens 124 be spaced the distance 170 apart can ensure that the distal tip 150 of the tensioning member 149 does not contact the cutting tip 141 of the endoscopic instrument 140 even when the distal tip 150 and the cutting tip 141 are located a distance distal to distal end 121 of the cap 120. For instance, in some embodiments, the working channel (e.g., working channel 114, as illustrated in FIG. 1A) can have an internal diameter of about 4.2 millimeters and the distance 170 can be a value (e.g., 0.1 millimeters) in a range from about 0.1 millimeters to about 0.5 millimeters.

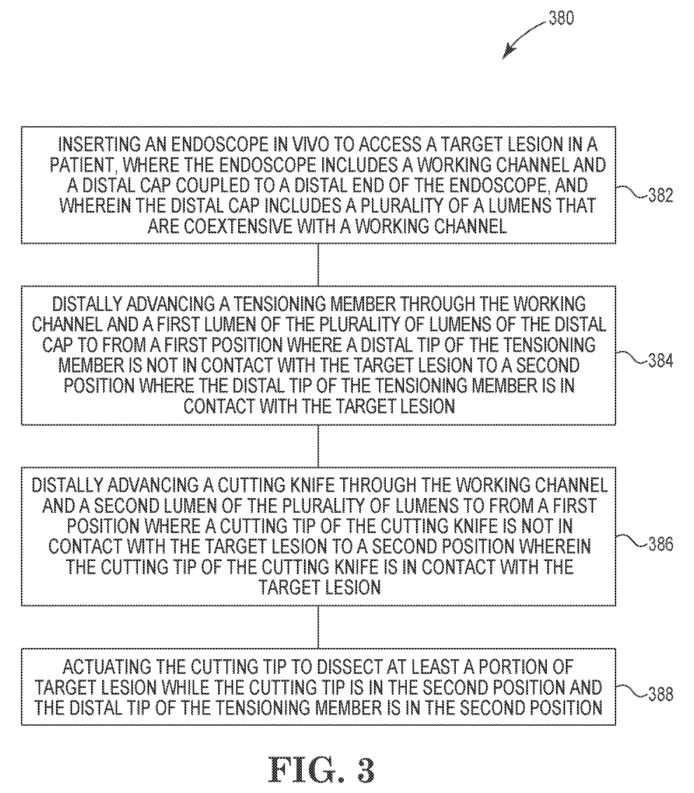

FIG. 3 provides an example of a method 380 of endoscopic submucosal dissection according to an embodiment of the disclosure. The method 380 can be employed with the endoscopes (e.g., endoscope 130) described herein. At 382, the method 380 can include inserting an endoscope in vivo to access a target lesion in a patient. For instance, the target lesion or target tissue can be located in an esophagus of a patient, as described herein.

As detailed herein, the endoscope includes a distal cap coupled to a distal end of the endoscope. The distal cap can include a plurality of a lumens that are coextensive with a working channel of the endoscope. In some embodiments, the endoscopes herein may include a cap (e.g., endoscopic cap, etc.) that is permanently or removably attachable (e.g., by a friction or interference fit, a threaded fit, a snap-lock fit, etc.) to the distal end of the endoscope. For instance, the method 380 can, in some embodiments, include affixing or attaching the cap to a distal end of the endoscope. In use, and by way of example, an endoscopic system or endoscope of the present disclosure may be assembled (e.g., prior to insertion into the patient) by attaching the cap to the distal end of the elongate tubular member e.g., endoscope. In some embodiments, the distal cap may be attachable either directly or indirectly to the endoscope by adhesives, soldering, welding, brazing, or mechanical attachments such as bands, clamps, or ratchet, or combinations thereof. However, in some embodiments the cap can be attached (e.g., is integral with) the endoscope.

At 384, the method 380 can include distally advancing a tensioning member (e.g., tensioning member) through the working channel and a first lumen of the plurality of lumens of the distal cap from a first position to a second position (where a distal tip of the tensioning member is in contact with the target lesion), as described herein. For instance, the tensioning member can be distally advanced by rotating a proximal knob a first direction to distally advance the distal tip of the tensioning member. Hence, a longitudinal position of the tensioning member can be altered via rotation of the knob in a given direction. For example, rotating the proximal knob in a first direction (e.g., clockwise) can move the distal tip forward (e.g., distally or in distal direction), while rotating the proximal knob in a second direction (e.g., counterclockwise) opposing the first direction can move the distal tip backward (e.g., proximally or in a proximal direction). In some embodiments, the knob may actuate the tensioning member to engage with the tissue (e.g., a target lesion) prior to and/or during a procedure.

At 386, the method 380 can include distally advancing a cutting knife through the working channel and a second lumen of the plurality of lumens from a first position to a second position (wherein a cutting tip of the cutting knife is in contact with the target lesion), as described herein. For instance, the cutting knife (or other type of endoscopic instrument) can be distally advanced by actuation of a handle, as described herein. In some embodiments, the handle may actuate the endoscopic instrument 140 to engage with the tissue prior to and/or during a procedure.

At 384, the method 380 can include actuating (e.g., rotating) the cutting tip (e.g., an electrocautery knife, resection tool, etc.) to dissect or cut at least a portion of target lesion. For instance, the dissection can occur while the cutting tip and the distal tip of the tensioning member are in contact with the target lesion. Hence, the dissection can occur while the distal tip of the tensioning member is imparting a force on the target lesion. Stated differently, the method 380 can include simultaneously or substantially simultaneously applying tension (via the tensioning member) and dissecting the target lesion (via the cutting member) in vivo.

As mentioned, dissecting the target lesion under tension (imparted by the distal tip of the tensioning member) can promote aspects herein e.g., promoting visibility of the target lesion, mitigating inadvertent contact with healthy tissue surrounding the target lesion, and/or promoting removal of the entire target lesion, etc. The portion of the target tissue engaged by the cutting tip may eventually separate or slough off and may be expelled/removed from within the patient by the body’s natural course. Alternatively, the medical professional may proximally retract the portion or all of the target tissue, for instance, via the cutting tip of the cutting member.

In some embodiments, the cutting knife and tensioning member can be incrementally or successive advanced distally. For instance, a portion of the target tissue or target lesion can be dissected (e.g., while under tension imparted by the tensioning member). In such instances, a remaining portion of the target lesion or target tissue may be located distal to the initial (e.g., first) position of the cutting knife and/or tensioning member. Hence, the cutting knife and the tensioning member can be advanced further distally to contact the remaining portion of the target lesion and can then dissect at least a portion of the remaining portion of the target tissue or target lesion (e.g., while under tension imparted by the tensioning member).

For example, subsequent to dissection of a portion of the target tissue a remainder of the target tissue may be located proximal to a position of the distal tip of the tensioning member and/or the cutting tip of the cutting knife. Hence, the proximal knob can be rotated (e.g., in the first direction) to distally advance the tensioning member (e.g., to a third position) that is distal to the first and second position of the distal tip, thereby permitting the distal tip to physically contact the remainder of the target lesion. In such instances, subsequent to advancing the tensioning member (e.g., to the third position), the handle can be actuated to distally advance the cutting tip of the cutting knife (e.g., from the second position to a third position), thereby permitting the cutting tip to contact with the remainder of the target lesion. In The method 380 can include actuating the cutting tip while the cutting tip and the distal tip are each in contact with the remainder of the target lesion.

The above described methodology of distally advancing the cutting tip of the cutting member and the distal tip of the tensioning member to contact and cut respective (e.g., longitudinal) portions of a target lesion can be continued until the entire target lesion or target tissue is dissected (e.g., is removed from the patient).

Hence, the endoscopes and methods of using the endoscopes herein can yield improve control over a degree of tension imparted in a target lesion (e.g., variable tensioning of a target lesion attributable at least in part to a movable longitudinal position of the tensioning member) and can ensure that the target lesion remains under tension prior to and during dissection by the cutting member of the endoscope, unlike previous approaches such as those the utilized clips and/or a MLTD.

Moreover, and unlike previous approaches such as those that employ MLTD and/or clips, the method 380 can include retracting the entire endoscope and all components associated with the method 380 subsequent to removal of the target lesion. For instance, the method 380 can include removal of each of the distal cap, the tensioning member, and the cutting knife from the patient after completing dissection of the target lesion. Thus, the endoscopes herein and methods of use thereof (e.g., method 380) can result in the effective removal (dissection) of the target lesion, and yet leaves no foreign materials (e.g., clips or MLTD) left in the patient after retracting the endoscope from the patient.

Although aspects of the present disclosure are described as associated with (e.g., attached to, mounted on, and/or extending through lumens therein) of a cap, and the above-described embodiments are described with reference to a cap element, it will be appreciated that the principles of the above disclosure are applicable to each embodiment even if a cap element is not provided. As such, references to a cap on a structure should be understood to include references to an element which is a part of (unitary or separate) the distal end of the structure. For instance, in various embodiments the tensioning member and cutting member or cutting instrument may be provided along or attached to / mounted on various surfaces of an endoscope with or without a cap 120 attached thereto.

In various embodiments, the endoscopic instrument of the present disclosure is not limited to a tissue cutting element (e.g., electrocautery knife, etc.), but may include a variety of medical instruments configured to manipulate a target tissue (e.g., ablative elements, needles or syringes configured to inject agents into the target tissue, etc.). In addition, it should be appreciated that various elements herein such as the tensioning member, the cap, and the endoscopic instrument of the present disclosure are not necessarily drawn to scale, but may be represented in a somewhat enlarged configuration to provide the requisite level of detail to understand and practice the embodiments of the present disclosure.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. An endoscope for tissue dissection, the endoscope comprising:
a cap attachable to a distal end of an endoscope having a working channel extending therethrough, the cap including lumens coextensive with the working channel;
a tensioning member disposable within the working channel and a first lumen of the lumens, wherein the tensioning member includes:
a distal tip; and
an actuation mechanism that is coupled to the distal tip and is configured to translate the distal tip longitudinally relative to at least the cap; and
an endoscopic instrument disposable within the working channel and a second lumen of the lumens.

2. The endoscope of claim 1, wherein a distal end of the distal tip is configured at an angle to a body portion of the distal tip.

3. The endoscope of claim 1, wherein the distal end of the distal tip is a pointed terminus.

4. The endoscope of claim 1, wherein the actuation mechanism is a screw mechanism, and wherein the endoscope further includes a knob coupled to a proximal end of the screw mechanism that is configured to rotate the screw mechanism.

5. The endoscope of claim 1, wherein the distal cap has a substantially planar distal end, and wherein the lumens extend through the substantially planar distal end of the distal cap.

6. The endoscope of claim 1, wherein the lumens extend in a substantially longitudinal direction entirely through the distal cap and are in fluid communication with the working channel.

7. The endoscope of claim 1, wherein the lumens each have a respective diameter that is less than a diameter of the working channel.

8. The endoscope of claim 1, wherein the lumens are uninterrupted circular lumens.

9. The endoscope of claim 1, wherein the distal tip is non-movably coupled to the actuation mechanism.

10. The endoscope of claim 1, wherein:
the distal tip is configured to translate longitudinally a first distance relative to a distal end of the distal cap, wherein the first distance is in a range from about 5 millimeters to about 15 millimeters; and
a cutting tip of the endoscopic instrument is configured to translate longitudinally a second distance from the distal end of the distal cap, wherein the second distance is in a range from about 5 millimeters to about 15 millimeters.

11. The endoscope of claim 1, wherein a diameter of the first lumen is substantially equal to a diameter of a proximal end the distal tip, a diameter of the actuation mechanism, or both.

12. The endoscope of claim 1, wherein a diameter of the second lumen is substantially equal to a diameter of a body portion of the endoscopic instrument.

13. The endoscope of claim 1, wherein the first lumen is spaced a distance from the second lumen.

14. The endoscope of claim 13, wherein the distance is in a range from about 0.1 millimeters to about 1.0 millimeters.

15. The endoscope of claim 13, wherein the working channel has an internal diameter of about 4.2 millimeters and wherein the distance is in a range from about 0.1 millimeters to about 0.5 millimeters.

16. An endoscope for tissue dissection, the endoscope comprising:
a distal cap attachable to a distal end of an endoscope having a working channel extending therethrough, the cap including lumens coextensive with the working channel, wherein the lumens include a first lumen and a second lumen that is spaced apart radially from the first lumen;
a tensioning member disposable within the working channel and a first lumen of the lumens, and wherein the tensioning member includes:
a distal tip including a distal end; and
a rotatable actuation mechanism that is non-movably coupled to a proximal end of the distal tip and is configured to rotate relative to a longitudinal axis of the endoscope to cause the distal tip to translate longitudinally relative to at least the distal cap; and
a cutting knife including a cutting tip, wherein the cutting knife is disposable within the working channel and a second lumen of the lumens.

17. A method of endoscopic tissue dissection, the method comprising:
inserting an endoscope in vivo to access a target lesion in a patient, wherein the endoscope includes a working channel and a distal cap coupled to a distal end of the endoscope, and wherein the distal cap includes a plurality of a lumens that are coextensive with a working channel of the endoscope;
distally advancing a tensioning member through the working channel and a first lumen of the plurality of lumens of the distal cap to from a first position where a distal tip of the tensioning member is not in contact with the target lesion to a second position where the distal tip of the tensioning member is in contact with the target lesion;
distally advancing a cutting knife through the working channel and a second lumen of the plurality of lumens to from a first position where a cutting tip of the cutting knife is not in contact with the target lesion to a second position wherein the cutting tip of the cutting knife is in contact with the target lesion; and
actuating the cutting tip to dissect at least a portion of target lesion while the cutting tip is in the second position and the distal tip of the tensioning member is in the second position.

18. The method of claim 17, further comprising:
rotating a proximal knob a first direction to distally advance the distal tip of the tensioning member to the first position; and
subsequent to actuating the cutting tip to cut at least a portion of target lesion, rotating the proximal knob the first direction to distally advance the tensioning member to a third position that is distal to the second position and is in contact with a remainder of the target lesion.

19. The method of claim 18, further comprising:
actuating a handle to distally advance the cutting tip of the cutting knife to the second position;
subsequent to advancing the tensioning member to the third position, actuating the handle to distally advance the cutting tip of the cutting knife from the second position to a third position that is distal to the second position; and
actuating the cutting tip while the cutting tip is in the third position and the distal tip of the tensioning member is in the third position to cut at least a portion of the remainder of the target lesion.

20. The method of claim 19, further comprising retracting the endoscope to remove the distal cap, the tensioning member, and the cutting knife from the patient after completing dissection of the target lesion, wherein substantially no materials are left in the patient after retracting the endoscope from the patient.
